(19) United States
(12) Patent Application Publication
(10) Pub. No.: US 2022/0054026 A1
(43) Pub. Date: Feb. 24, 2022

(54) MOBILE PULSE OXIMETRY AND ECG ELECTRODE TELEMETRY DEVICE, SYSTEM AND METHOD OF USE

(71) Applicant: Darin Slack, Corona, CA (US)

(72) Inventor: Darin Slack, Corona, CA (US)

(21) Appl. No.: 17/210,972

(22) Filed: Mar. 24, 2021

Related U.S. Application Data
(60) Provisional application No. 63/068,088, filed on Aug. 20, 2020.

(51) Int. Cl. A61B 5/0205; A61B 5/00; A61B 5/021; A61B 5/274; A61B 5/318; A61B 5/08; A61B 5/145

(52) U.S. Cl. CPC A61B 5/02055; A61B 2560/0214; A61B 5/742; A61B 5/6824; A61B 5/6833; A61B 5/6814; A61B 5/6823; A61B 5/021; A61B 5/7264; A61B 5/274; A61B 5/6828; A61B 5/318; A61B 5/7214; A61B 5/0816; A61B 5/14542; A61B 2562/0219; A61B 5/002

(57) ABSTRACT

A multiparameter monitor (MPM) for a patient includes a disposable electrode patch (DEP) comprising a plurality of male snap post electrodes, an optics interface and a thermal/respiratory interface to a skin surface of the patient. The MPM also includes an electronics module and wireless transmitter (EMT) in connection with a plurality of female snap receptors and configured to transmit a signal data based on a connection and a disconnection of the plurality of the female snap receptors to the male snap post electrodes. The MPM additionally includes a reflective pulse oximeter (RPO) separated from a skin surface of the patient based on a thickness of the DEP, the RPO in communication with the wireless transmitter. The MPM further includes a mobile photoplethysmogram processor (PPG) in communication with the wireless transmitter and the optics interface and configured to filter a motion artifact in the PPG.

### CROSS-REFERENCE TO RELATED APPLICATIONS

This application claims the benefit of the priority date of earlier filed U.S. Provisional Utility Application Ser. No. 63/068,088 filed Aug. 20, 2020 for Darin Slack under the same title and incorporated herein by reference in its entirety.

### BACKGROUND AND FIELD OF INVENTION

Pulse oximetry is a noninvasive method for monitoring a person's oxygen saturation. Though its reading of peripheral oxygen saturation (SpO2) is not always identical to the more desirable reading of arterial oxygen saturation (SaO2) from arterial blood gas analysis, the two are correlated well enough that the safe, convenient, noninvasive, inexpensive pulse oximetry method is valuable for measuring oxygen saturation in clinical use.

In its most common transmissive application mode, a sensor device is placed on a thin part of the patient's body, usually a fingertip or earlobe, or in the case of an infant, across a foot. The device passes two wavelengths of light through the body part to a photodetector. It measures the changing absorbance at each of the wavelengths, allowing it to determine the absorbances due to the pulsing arterial blood alone, excluding venous blood, skin, bone, muscle, fat, and (in most cases) nail polish.

In-patient health care for cardiac symptom diagnosis includes ECG and EKG analysis via electrode patches applied to a patient's skin near the heart. An Einthoven triangle is established by applying an electrode patch near the hip, preferably over non-musculature and another two electrode patches are applied to the chest. Multiple electrode patches help to establish where an ECG signal originates, which direction it is traveling and to establish a common ‘ground.’ Therefore it is common to apply 8, 12 and even 18 electrode patches to a patient who is non-ambulatory and wired to the diagnostic equipment. The multiple electrodes are commonly color coded in order to assist in establishing signal direction and ground.

Conventionally, the electrodes are connected by dedicated wires straight to the diagnostic equipment but getting from the patient even to a bedside piece of diagnostic equipment requires at least several feet of wires. However, leads can act as antennae for noise and produce artifacts of the desired signals. Lead artifacts distort a biological signal and must be filtered or ignored in the diagnostic process. Minimizing artifacts therefore becomes a priority in signal integrity and signal processing at the receiver. It is therefore desirable to minimize the lead wires to the multiple electrodes for cleaner signals and more accurate diagnostics.

Out-patient services cannot connect the electrode lead wires directly to the diagnostic equipment since it is not practical for a patient to carry the diagnostic equipment around with them. Therefore, a transceiver worn on the patient's wrist or carried in a pocket receives the multiple leads from the multiple electrode patches and communicates with the diagnostic equipment. However, this does not solve the lead artifact issues though it may shorten the lead wires from the electrode patches to the receiver carried with the patient.

Standard snap leads are a convenient and quick way of hooking a patient up to a diagnostic piece of equipment. However, it is also common for the electrode patches to come off the skin due to the leads pulling on them in outpatient everyday use and in-patient movements. This loss of contact results in loss of telemetry and exposes the patient to downtime and risks an unmonitored cardiac event in the interim time period(s). Also, when a patient takes a shower the lead wires are usually detached from the electrode patches because the receiver is not waterproof. This also exposes the patient to unattended telemetry downtime.

Cell phone computing power is providing many opportunities for analyzing telemetry data. What required a server or specialized equipment in the past may now be at least conceptualized on cell phone technology. However, medical grade telemetry and analysis is not yet available through cell phones alone. There is therefore a long felt need for a device and method to minimize lead wires from an electrode patch and allow a patient to take a shower and go about normal life as much as possible that has gone unmet until the present Applicants' disclosure.

### SUMMARY OF THE INVENTION

A multiparameter monitor (MPM) for a patient includes a disposable electrode patch (DEP) comprising a plurality of male snap post electrodes, an optics interface and a thermal/respiratory interface to a skin surface of the patient. The MPM also includes an electronics module and wireless transmitter (EMT) in connection with a plurality of female snap receptors and configured to transmit a signal data based on a connection and a disconnection of the plurality of the female snap receptors to the male snap post electrodes. The MPM additionally includes a reflective pulse oximeter (RPO) separated from a skin surface of the patient based on a thickness of the DEP, the RPO in communication with the wireless transmitter. The MPM further includes a mobile photoplethysmogram processor (PPG) in communication with the wireless transmitter and the optics interface and configured to filter a motion artifact in the PPG.

A method for a MPM monitoring of a patient includes, disposing a disposable electrode patch (DEP) comprising a plurality of male snap post electrodes, an optics interface and a thermal interface to a skin surface of the patient. The method also includes connecting an electronics module and wireless transmitter (EMT) with a plurality of female snap receptors and configured to transmit a signal data based on a connection and a disconnection of the plurality of the female snap receptors to the male snap post electrodes. The method additionally includes separating a reflective pulse oximeter (RPO) from a skin surface of the patient based on a thickness of the DEP, the RPO in communication with the wireless transmitter. The method further includes filtering a motion artifact in a photoplethysmogram via a processor (PPG) in communication with the wireless transmitter and the optics interface.

A number of discrete electrode telemetry devices comprise a single female snap receptor configured in a leadless body of a single discrete electrode sensor device. The single female snap receptor attaches to a single male snap post of a single discrete standard circular disposable electrode patch, wherein the sensor comprises a plurality of plug-in interface ports on the leadless body thereof. A multiplexor circuit is engineered to multiplex and combine a plurality of signal data collected from the interface ports as a piecewise output thereof. A wireless transmitter module is disposed in connection with the single female snap receptor, the wireless transmitter configured to transmit the piecewise multiplexed and combined signal data based on a connection and a disconnection of the single female snap receptor thereto. Furthermore, a spooled memory stores and relays to a smart phone the plurality of signal data for a multiplexed and a combined piecewise transmission from the wireless transmitter module.

A telemetry system comprises an Nth plurality of button-like wireless and leadless discrete electrode sensor devices and an equal plurality of standard circular disposable electrode patches, wherein a single female snap receptor is configured in a leadless body of each of the discrete electrode sensor devices, each female snap receptor configured to attach to a single male snap post of the plurality of standard circular disposable electrode patches. Also, a multiplexor circuit is configured to multiplex and combine a plurality of signal data collected from a plurality of interface ports on each leadless discrete electrode telemetry device as a composite output thereof. Additionally, a wireless transmitter module is disposed in connection with each female snap receptor, each respective wireless transmitter module configured to transmit a signal in reference to an electrical ground thereof to a receiver. Furthermore, a wireless receiver module is configured to receive and to process an Nth plurality of transmitted signals at a smart phone from the Nth plurality of discrete electrode telemetry devices into an Nth−1 number of signals greater than zero. A reference cluster is also included, the cluster comprising the plug-in interface ports on each discrete electrode sensor device and a plurality of reference electrodes connected therein, wherein a reference electrode of the reference cluster is shared by any number of sensors.

A disclosed method for telemetry comprises providing an Nth plurality of wireless and leadless discrete electrode telemetry devices and an equal plurality of standard circular disposable electrode patches attachable to a patient, wherein a single female snap receptor is configured in a leadless body of each of the discrete electrode telemetry devices, each female snap receptor configured to attach to a single male snap post of the plurality of standard circular disposable electrode patches. The method also comprises multiplexing and merging a plurality of signal data collected from a plurality of interface ports on each leadless discrete electrode telemetry device as an output thereof. The method additionally comprises transmitting a multiplexed and a merged signal in reference from the respective female snap receptor to a remote receiver via a wireless transmitter processor disposed in connection with each female snap receptor. The method further comprises receiving and processing an Nth plurality of the transmitting signals at a smart phone from the Nth plurality of discrete electrode telemetry devices into an Nth−1 number of signals greater than zero via a receiver processor. The method yet comprises spooling a memory configured to store and relay the plurality of signal data for a multiplexed and a merged piecewise transmission from the wireless transmitter module based on a connection and a disconnection of the single female snap receptor to the wireless transmitter module and the ground thereof.

Other aspects and advantages of embodiments of the disclosure will become apparent from the following detailed description, taken in conjunction with the accompanying drawings, illustrated by way of example of the principles of the disclosure.

### BRIEF DESCRIPTION OF THE DRAWINGS

FIG. 1 is a block diagram of the components of the mobile pulse oximeter and ECG electrode telemetry device and system in accordance with an embodiment of the present disclosure.

FIG. 2 is a block diagram of the components of the mobile pulse oximeter and ECG electrode telemetry device and system with a thermal interface in accordance with an embodiment of the present disclosure.

FIG. 3 is a block diagram of the components of the mobile pulse oximeter and ECG electrode telemetry device and system with thermal and optical interfaces in accordance with an embodiment of the present disclosure.

FIG. 4 is a top view of the disposable electrode patch (DEP), disposable medical grade electrode with a thermal and respiratory interface in accordance with an embodiment of the present disclosure.

FIG. 5 are side view depictions of the DEP and the EMT (electronics module transmitter with a thermal and respirator interface in accordance with an embodiment of the disclosure.

FIG. 6 are side view depictions of the DEP and the EMT (electronics module transmitter with a thermal and respirator interface and offset snap receptors in accordance with an embodiment of the disclosure.

FIG. 7 is a top view of the disposable electrode patch (DEP), disposable medical grade electrode with a thermal and respiratory interface and a multipin connector in accordance with an embodiment of the present disclosure.

FIG. 8 are side view depictions of the DEP and the EMT (electronics module transmitter with a thermal and respirator interface and multipin connector interfaces in accordance with an embodiment of the disclosure.

FIG. 9 is a depiction of an angle of incidence and angle of reflection based on a thickness of the DEP for a reflexive pulse oximeter reading in accordance with an embodiment of the present disclosure.

FIG. 10 is a block diagram of a method for a MPM ambulatory monitoring including ECG, SpO2 and thermal/respiratory functions in accordance with an embodiment of the present disclosure.

FIG. 11 depicts reflective PPG waveforms for a finger, forehead, ear and wrist in accordance with an embodiment of the present disclosure.

FIG. 12 depicts reflective PPG waveforms for an upper sternum, upper breast, lower breast and lower sternum in accordance with an embodiment of the present disclosure.

FIG. 13 depicts a placement of the disclosure on the human body above the sternum parallel to a chest cleavage in accordance with an embodiment of the present disclosure.

FIG. 14 depicts a top perspective view of the reusable snap dome switch and electronics case disconnected from the disposable sensors and case in accordance with an embodiment of the present disclosure.

FIG. 15 depicts an exploded view of components of the reusable snap dome switch and electronics case connected with the disposable sensors and case in accordance with an embodiment of the present disclosure.

FIG. 16 depicts a visual display of a correlated timeline data from six sensors indicating multiple irregularities outside normal events that coincide to determine a patient's health in accordance with an embodiment of the present disclosure.

FIG. 17 depicts a top perspective of the reusable snap dome switch and electronics case connected with the disposable sensors and case in accordance with an embodiment of the present disclosure.

FIG. 18 depicts a bottom perspective of the reusable snap dome switch and electronics case connected with the disposable sensors and case in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Reflectance pulse oximetry is a less common alternative to transmissive pulse oximetry. This method does not require a thin section of the person's body and is therefore well suited to a universal application such as the feet, forehead, and chest, but it also has some limitations. Vasodilation and pooling of venous blood in the head due to compromised venous return to the heart can cause a combination of arterial and venous pulsations in the forehead region and lead to spurious SpO2 results. Such conditions occur while undergoing anesthesia with endotracheal intubation and mechanical ventilation or in patients in the Trendelenburg position.

Motion artifact can be a significant limitation to pulse oximetry monitoring resulting in frequent false alarms and loss of data. This is because during motion and low peripheral perfusion, many pulse oximeters cannot distinguish between pulsating arterial blood and moving venous blood, leading to underestimation of oxygen saturation. Early studies of pulse oximetry performance during subject motion made clear the vulnerabilities of conventional pulse oximetry technologies to motion artifact.

Throughout the present disclosure, the term ‘piecewise’ refers to a transmission of multiplexed and superimposed signal data (also called a piecewise function or a hybrid function) and a transmission which is defined by multiple sub-data, each sub-data applying to a certain time interval of the complete transmission domain (a sub-domain) in analog or digital formats. The term ‘merged,’ ‘composite,’ ‘combined,’ and ‘superimposed’ refer to a number of signals that have been made into a single signal. The component signals thereof may be analog or digital and the piecewise transmission may therefore include a respective time period for the analog and the digital pieces thereof. The term ‘sensor’ refers to disclosed components that may include an electrode patch, an amplifier, a transmitter and any other components associated with the disclosed telemetry device in transmitting, detecting, converting and relaying signals from the human body to electric and electronic systems and devices. Also, as used in the present disclosure, the term ‘single’ refers to a solitary electrode patch and telemetry unit as opposed to prior art including multiple patches and or multiple electrodes or multiple telemetry devices. Additionally, the term ‘multiplexer’ refers to a device that selects one of several analog or digital input signals and forwards the selected input into a single line. A multiplexer of 2 inputs has n select lines, which are used to select which input line to send to the output. The term ‘spooled memory,’ refers to a first in last out memory capable of real time compression and magnitude scaling for efficient transmission. The term ‘electrode patch’ as used in the present disclosure includes an electrical conductor used to make contact with a nonmetallic part of a circuit where a patient is the start of a circuit in accordance with an embodiment of the present disclosure. The patch component is a standard disposable patch with sticky adhesive applied to an underside as is standard in the art of electrode patches. The telemetry device disclosed therefore is not an electrode nor an electrode patch but a novel and unobvious device for the quick and convenient gathering of telemetry information from an ambulatory patient via wireless transmissions over radio waves or ultrasonic waves and any other types of communicable waveforms. The term ‘electrical ground’ refers to a voltage reference over which a signal level is taken or measured and that provides a return for current.

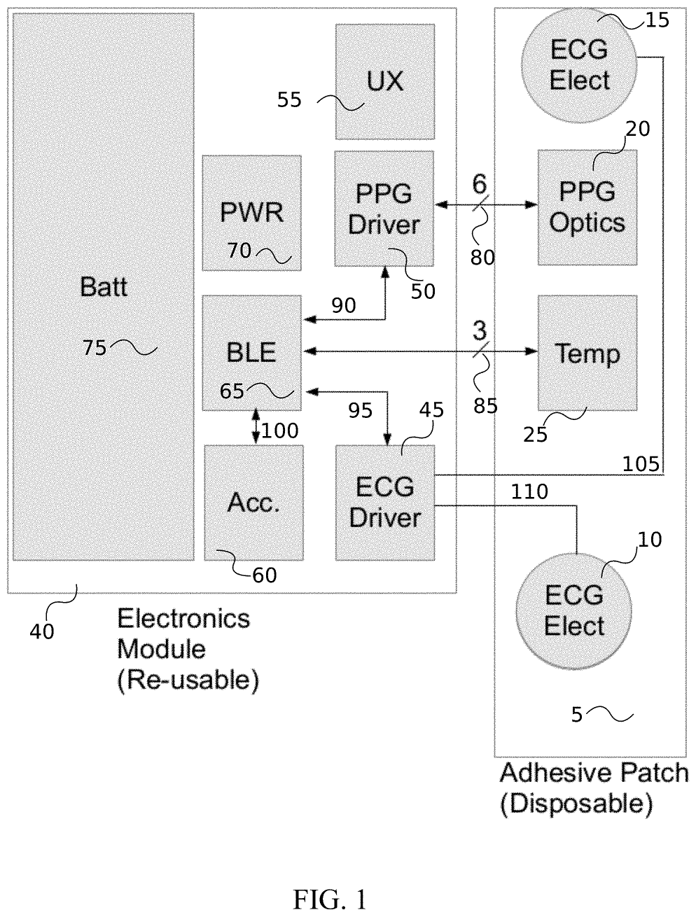

FIG. 1 is a block diagram of the components of the mobile pulse oximeter and ECG electrode telemetry device and system in accordance with an embodiment of the present disclosure. The depiction includes the DEP 5, a first ECG electrode 10, a second ECG electrode 15, a PPG Optics 20, a temperature and respiratory interface 25, an EMT 40, an ECG driver 45, a PPG driver 50, a user experience interface 55, an accelerator 60, a BLE 65, a power supply 70 and a battery 75, a 6 conductor optics bus 80, a 3 conductor temperature and respiratory bus 85, a PPG driver conductor 90, an ECG driver conductor 95, an accelerator to BLE conductor 100, an ECG driver to the second ECG electrode conductor 105 and a conductor 110 from the ECG driver to the first ECG electrode.

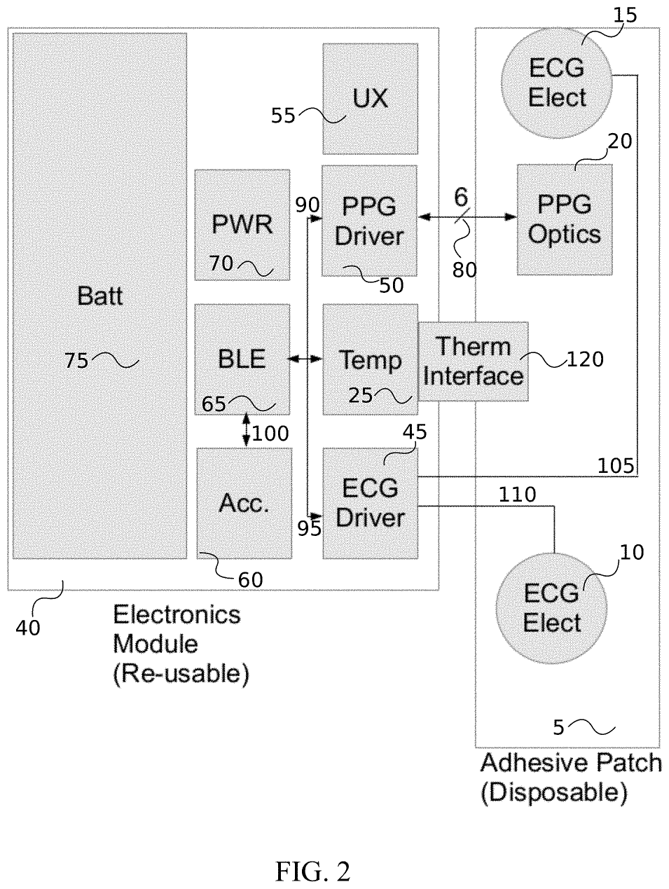

FIG. 2 is a block diagram of the components of the mobile pulse oximeter and ECG electrode telemetry device and system with a thermal interface in accordance with an embodiment of the present disclosure. The depiction includes many of the blocks, buses and conductors of FIG. 1 with same reference numbers and reference lines but in addition includes a thermal interface 120. The thermal interface is synergistic with a respiratory interface for measuring, monitoring and telemetry of respiratory functions including breath rate, inhale, exhale parameters and audio thereof.

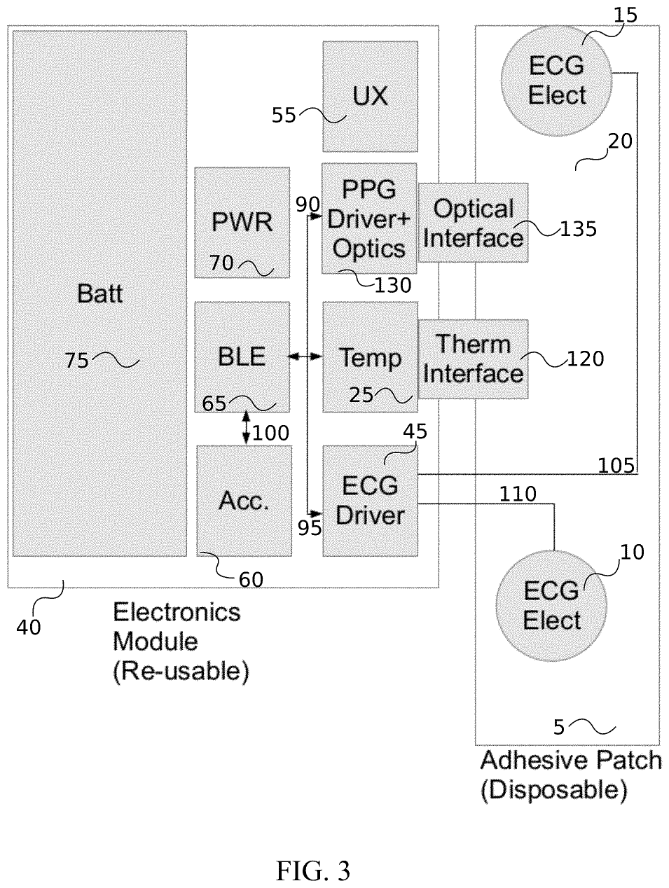

FIG. 3 is a block diagram of the components of the mobile pulse oximeter and ECG electrode telemetry device and system with thermal and optical interfaces in accordance with an embodiment of the present disclosure. The depiction includes many of the blocks, buses and conductors of FIG. 1 and FIG. 2 with same reference numbers and reference lines but in addition includes a PPG driver and optics driver 130 and an optical interface 135 for the reflective pulse oximeter.

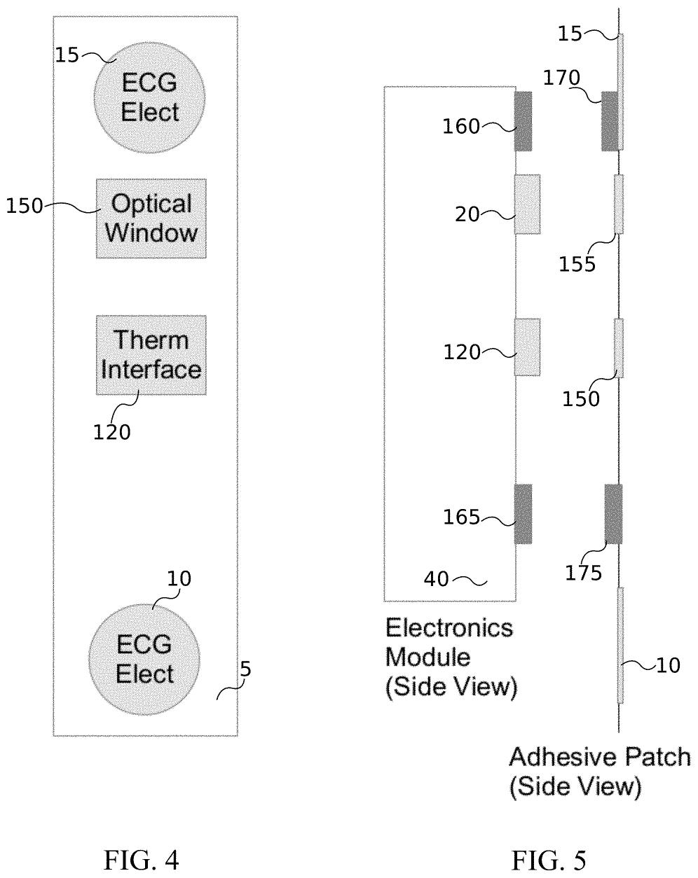

FIG. 4 is a top view of the disposable electrode patch (DEP), disposable medical grade electrode with a thermal and respiratory interface in accordance with an embodiment of the present disclosure. The depiction includes the DEP 5, first ECG electrode 10, the second ECG electrode 15, the optical window 150 for placing over an upper sternum, and the thermal interface 120.

FIG. 5 are side view depictions of the DEP and the EMT (electronics module transmitter with a thermal and respirator interface in accordance with an embodiment of the disclosure. The depiction includes the EMT 40, the first ECG electrode 10, the second ECG electrode 15, the PPG optics module 20, the thermal interface connect module 120, the optical window interface 155, the thermal interface window 150, and the ECG snap electrodes 160 and 165.

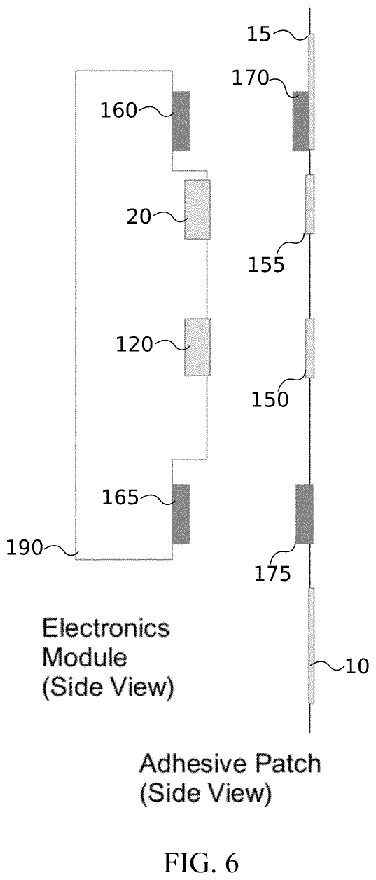

FIG. 6 are side view depictions of the DEP and the EMT (electronics module transmitter with a thermal and respirator interface and offset snap receptors in accordance with an embodiment of the disclosure. The depiction includes the offset EMT 190, the first ECG electrode 10, the second ECG electrode 15, the PPG optics module 20, the thermal interface connect module 120, the optical window interface 155, the thermal interface window 150, and the offset ECG snap electrodes 160 and 165.

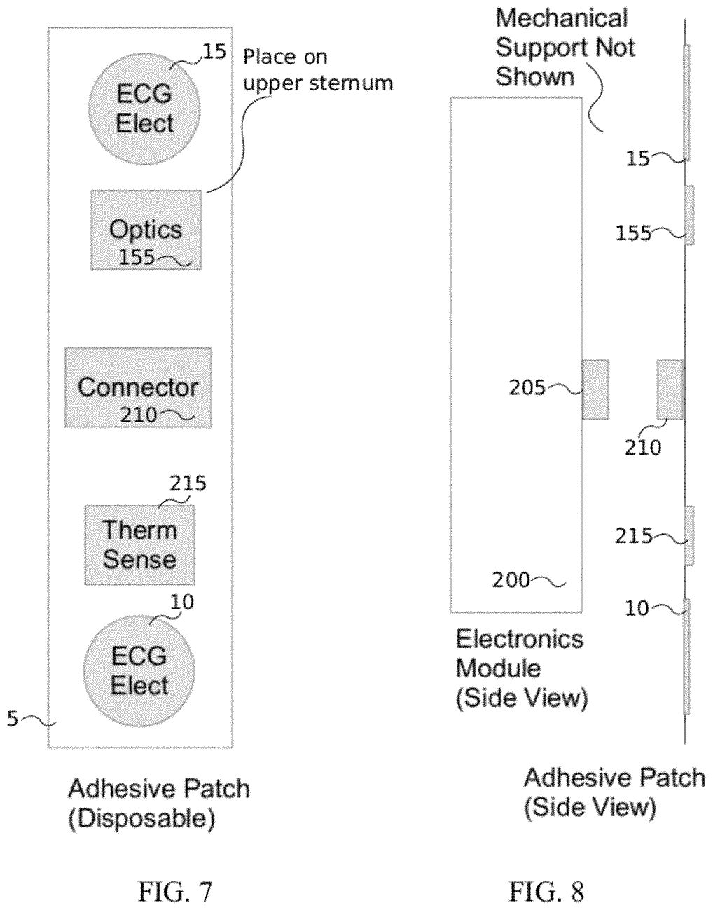

FIG. 7 is a top view of the disposable electrode patch (DEP), disposable medical grade electrode with a thermal and respiratory interface and a multipin connector in accordance with an embodiment of the present disclosure. The depiction includes the DEP 5, first ECG electrode 10, the second ECG electrode 15, the optics 155 for placing over an upper sternum, and the thermal sensor 215 and the connector 210.

FIG. 8 are side view depictions of the DEP and the EMT (electronics module transmitter with a thermal and respirator interface and multipin connector interfaces in accordance with an embodiment of the disclosure. The depiction includes the EMT 200, the first ECG electrode 10, the second ECG electrode 15, the PPG optics 155, the thermal and respirator sensor 215, and the ECG snap electrodes 10 and 15 and the multipin connectors 205 and 210.

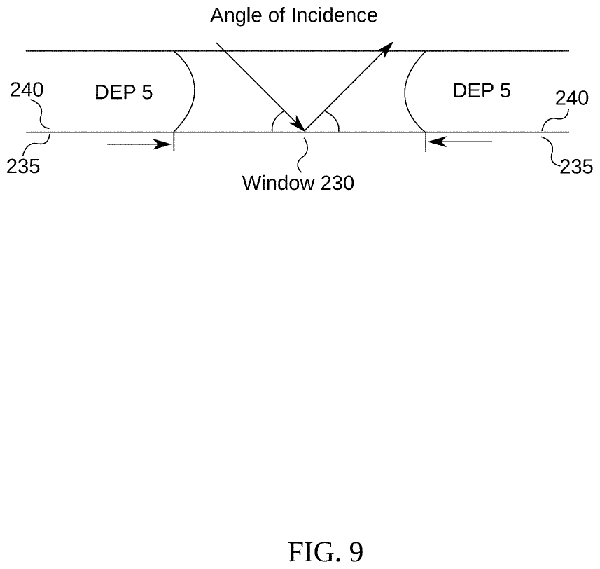

FIG. 9 is a depiction of an angle of incidence and angle of reflection based on a thickness of the DEP for a reflexive pulse oximeter reading in accordance with an embodiment of the present disclosure. The depiction includes the DEP 5 defining a window 230 to the patient's skin 235, an underside 240 of the DEP adjacent the patient's skin and the angle of incidence theta and the angle of reflection delta. A thickness of the DEP 5 is given by the extremes of the convexity on the outside edges of the DEP 5.

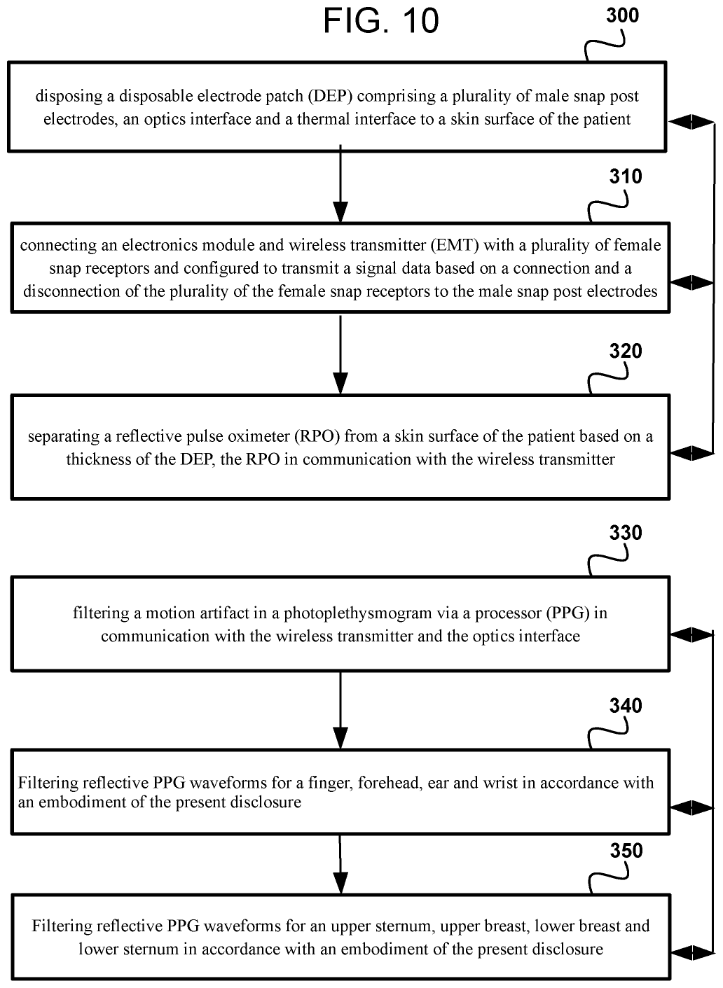

FIG. 10 is a block diagram of a method for a MPM ambulatory monitoring including ECG, SpO2 and thermal/respiratory functions in accordance with an embodiment of the present disclosure. The block diagram includes disposing 300 a disposable electrode patch (DEP) comprising a plurality of male snap post electrodes, an optics interface and a thermal interface to a skin surface of the patient. The method also includes connecting 310 an electronics module and wireless transmitter (EMT) with a plurality of female snap receptors and configured to transmit a signal data based on a connection and a disconnection of the plurality of the female snap receptors to the male snap post electrodes. The method additionally includes separating 320 a reflective pulse oximeter (RPO) from a skin surface of the patient based on a thickness of the DEP, the RPO in communication with the wireless transmitter. Embodiments of the method further include filtering 330 a motion artifact in a photoplethysmogram via a processor (PPG) in communication with the wireless transmitter and the optics interface. The embodied methods also include filtering 340 reflective PPG waveforms for a finger, forehead, ear and wrist in accordance with an embodiment of the present disclosure. The embodied methods further include filtering 350 reflective PPG waveforms for an upper sternum, upper breast, lower breast and lower sternum in accordance with an embodiment of the present disclosure.

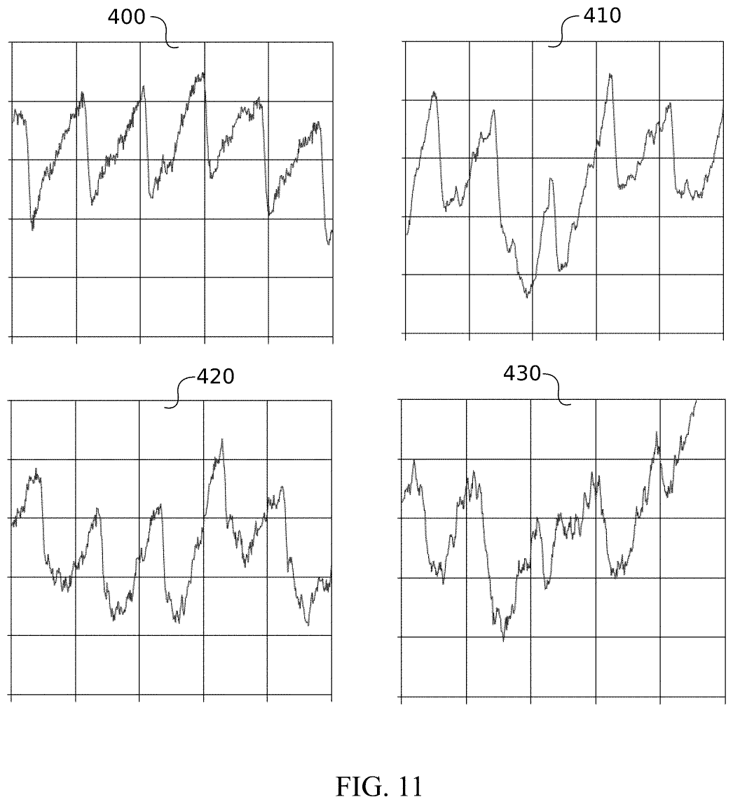

FIG. 11 depicts reflective PPG waveforms for a finger, forehead, ear and wrist in accordance with an embodiment of the present disclosure. The depiction includes the finger PPG 400, the forehead PPG 410, the ear PPG 420 and the wrist PPG 430. Therefore, the disclosure discriminates a location on a patient's body for an application of the MPM from a difference in critical parameters including pulse, respiration, temperature and blood volume at that location and distinguishes over the prior art. Waveforms at rest and while ambulatory are also obtained, stored in memory and compared against each other in an evaluation of the critical parameters.

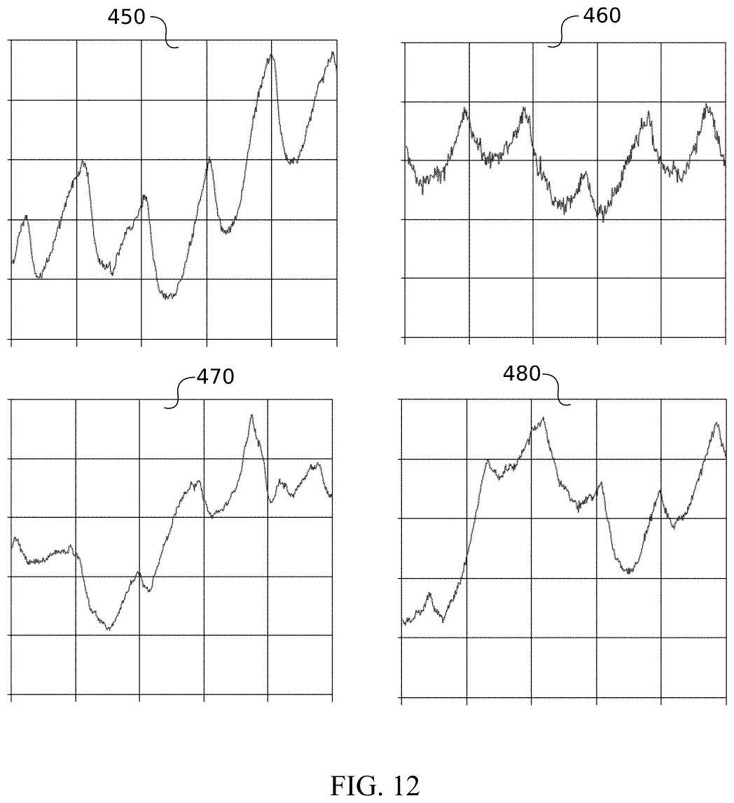

FIG. 12 depicts reflective PPG waveforms for an upper sternum, upper breast, lower breast and lower sternum in accordance with an embodiment of the present disclosure. The depiction includes the upper sternum PPG 450, the upper breast PPG 460, the lower breast PPG 470 and the lower sternum PPG 480. Therefore, the disclosure discriminates a location on a patients body for an application of the MPM from a difference in critical parameters including pulse respiration, temperature and blood volume at that location and distinguishes over the prior art. Waveforms at rest and while ambulatory are also obtained, stored in memory and compared against each other in the evaluation of the critical parameters.

Embodiments of the Spo2 sensor are engineered to detect the oxygen saturation and blood pressure from the chest area, specifically above the sternum area. PPG is utilized for both sensors.

Visual correlation of the collected data is utilized on a timeline graph in such a way to determine multiple possible irregularities or outside the normal events. These out of the normal parameters are visualized in the software to show possible indications that coincide with each other to determine a patient's health.

Embodiments of the disclosure include the Spo2 and blood pressure sensors and sensing from the chest area as well as the correlation data that could indicate a health concern and a pattern or series of abnormal bio-readings that correlate with each other or even rule out a correlation. With the correlation data overall patient health can be more identified quickly and accurately.

### ECG-Button System Overview

The ECG-Button System utilizes six sensors adjacent a sternum area of a chest via a wireless remote monitoring system intended for use by healthcare professionals for continuous collection of physiological data in home and healthcare settings. This can include heart rate, electrocardiography (ECG), respiratory rate, skin temperature, activity and posture (body), SpO2 and Blood pressure. Data are transmitted wirelessly from the ECG-Button Sensor for storage and analysis. The ECG-Button System can include the ability to notify healthcare professionals when physiological data fall outside selected parameters.

The ECG-Button System is an ECG acquisition, storage, and transmission devices that utilizes disposable adhesive to maintain contact with the patient's skin and a hydrogel to promote electrical connectivity. The Platform has a re-usable sensor/recorder module allowing collection, storage and transfer of physiological data such as electrocardiogram (ECG), temperature, respiratory, SpO2 and blood pressure. The system application allows healthcare professionals to use Software Library algorithm located on the server for aided analysis and review; Users can only view the data on the intended device (such as a mobile phone or computer).

A user may view the physiological data in real time from the smart phone (or similar devices) while it records the data. The ECG-Button System is comprised of the following sub-systems:

### ECG-Button Sensor

The Sensor includes an Adhesive Patch and a reusable Sensor Module attached to the patch. The Patch is designed as a disposable self-adhesive interface to the body. The Sensor Module performs processing functions related to capture of physiologic data and also performs bidirectional communication with the relay device.

### Adhesive Patch

The ECG-Button Adhesive Patch is a single-use, disposable adhesive patch, which includes two ECG electrodes, temperature measuring device, zinc air battery, and a flexible circuit and data collectors. The proposed and predicate Adhesive Patch were designed and constructed with similar multilayers of material and adhesives. Both patches include a skin adhesive side on its base. Both patches have the two ECG electrodes on the adhesive (skin-contact) side of the patch. The electrodes on both patches are covered with a disc of hydrogel. Both patches are powered by a zinc air battery. The proposed Adhesive Patch is different than the predicate in patch outer perimeter.

The patch is the only component in the device that comes in contact with skin. The skin-contact materials of the proposed device are identical to those of the predicate.

### Sensor Module

The Sensor Module performs processing functions related to capture of physiologic data and also performs bi-directional communication. The Sensor Module includes the embedded processor, tri-axial accelerometer, and Bluetooth low energy transceiver. The Sensor Module is designed to function only when inserted into the disposable patch cavity. After the insertion, the Sensor Module is sealed within the cavity by the patch liner and does not have direct contact with the user.

The hardware of the Sensor Module of the disclosed device is identical to that of the primary predicate. The disclosed firmware is included in the Sensor Module.

The ECG-Button System was developed with an Application Programming Interface intended to allow development of user interface applications enabling healthcare professionals to access collected vital information. A comparison of the indications for use statements for the predicate and the proposed device are provided herein. Under the current indication, the product is configured with deployment of both the Relay Software and the ECG-Button System Server Library. The data flow from Sensor to Relay and continue to be transmitted to the ECG-Button System Server for storage and analysis.

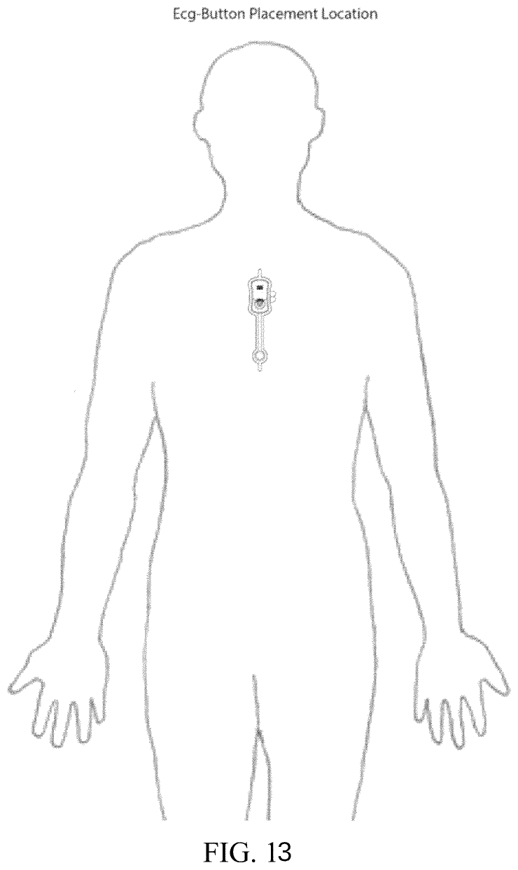

FIG. 13 depicts an ECG-Button placement location of the disclosure on the human body above the sternum parallel to a chest cleavage in accordance with an embodiment of the present disclosure.

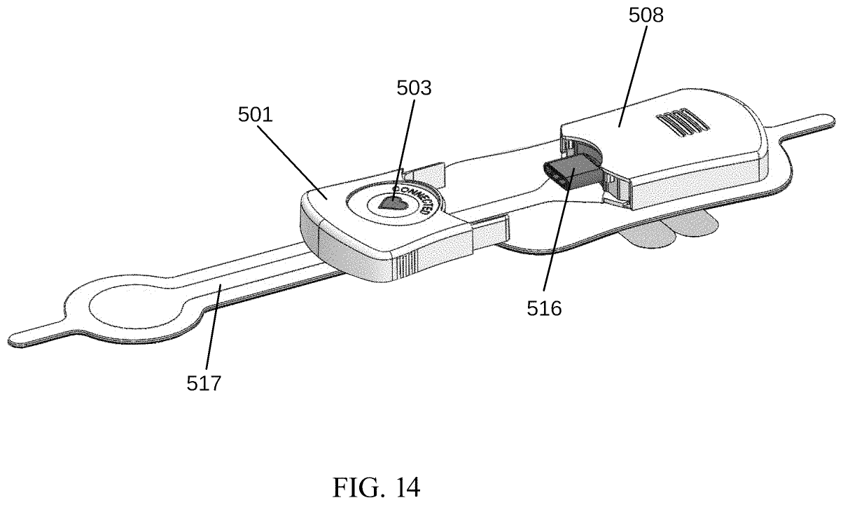

FIG. 14 depicts a top perspective view of the reusable snap dome switch and electronics case disconnected from the disposable sensors and case in accordance with an embodiment of the present disclosure. The view includes the case 508 over the disposable adhesive patch, the snap dome switch or button 503, the case 501 over the reusable sensor module, the cardio flex circuit 517 and the USB male connector 516.

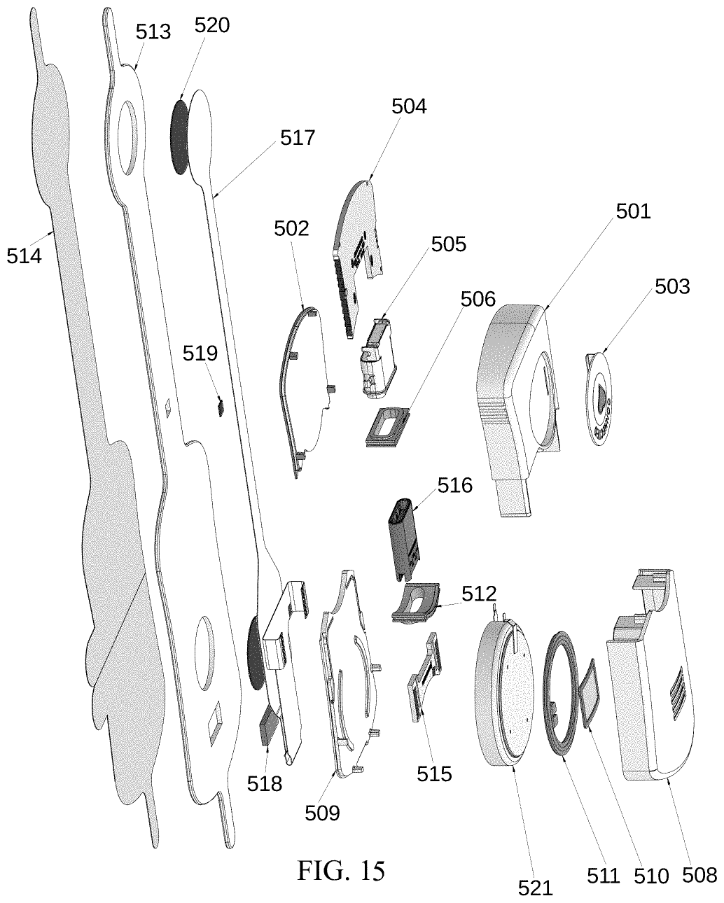

FIG. 15 depicts an exploded view of components of the reusable snap dome switch and electronics case connected with the disposable sensors and case in accordance with an embodiment of the present disclosure. The view includes the case 501 over the reusable sensor module, the case cover master 502, the snap dome switch or button 503, the main printed circuit board 504 for the reusable sensor module, the USB receiving connector 505, the USB connector seal 506, the master case 508 for the adhesive diposable patch, the case cover 509, the EPTFE oleophobic membrane 510, the battery top seal 511, the male USB connector seal 512, the master patch 513, the master cover patch 514, the battery printed circuit board 515, the male USB connector 516, the Versa Cardio flex circuit 517, the optical sensor 518, the digital temperature sensor 519, the ECG electrode 520, and the battery 521.

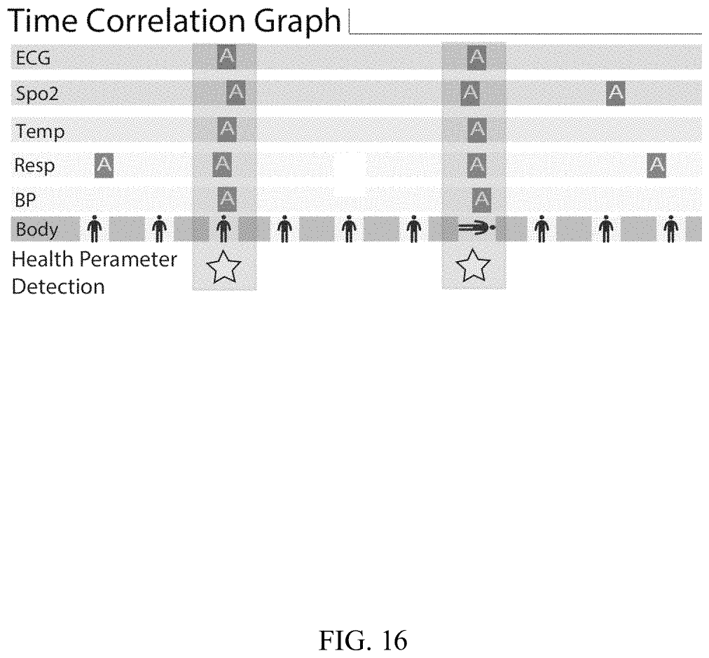

FIG. 16 depicts a visual display of a correlated timeline data from six sensors indicating irregularities outside normal events that coincide to determine a patient's health in accordance with an embodiment of the present disclosure. The body position axis shows a standing position and a laying down position and associated 5 data for ECG, Spo2, body Temperature, Respiration and blood pressure. A Health Parameter detection axis indicates normal or abnormal alignment of results in near real time. Each abnormal or out of normal range indicator or a combination of those indicators can be an alarm or a confirmation of health concerns.

Body position is measured by an accelerometer including the effects of gravity and dynamic acceleration and thus allowing a determination of a patient's body orientation and movements on collected and telemetered data. Blood pressure BP is the pressure of circulating blood against the walls of blood vessels. BP outside a normal range can indicate a health concern. Body temperature is a personal datum regarding the health status of an individual and is characterized by volatility and temporality. The personal nature of this information results from how and where it originates in the body. Body temperature outside a normal range can indicate a health concern.

The ECG-Button device and software utilizes six sensors to capture, display and correlate ECG, Spo2 (blood oxygen level), body temperature, Respiratory rate, Blood pressure and body position from the sternum area of the chest. These six sensors display the collected data and mark abnormal or out of normal range results in near real time along a timeline, the time line can be minutes, hours, days, months or even years. The disclosed graph displays the sensor data along the time line. The time alignment of abnormal or out of range results can be an indicator of a health concern. Each abnormal or out of normal range indicator or a combination thereof is an indicator of a health concern.

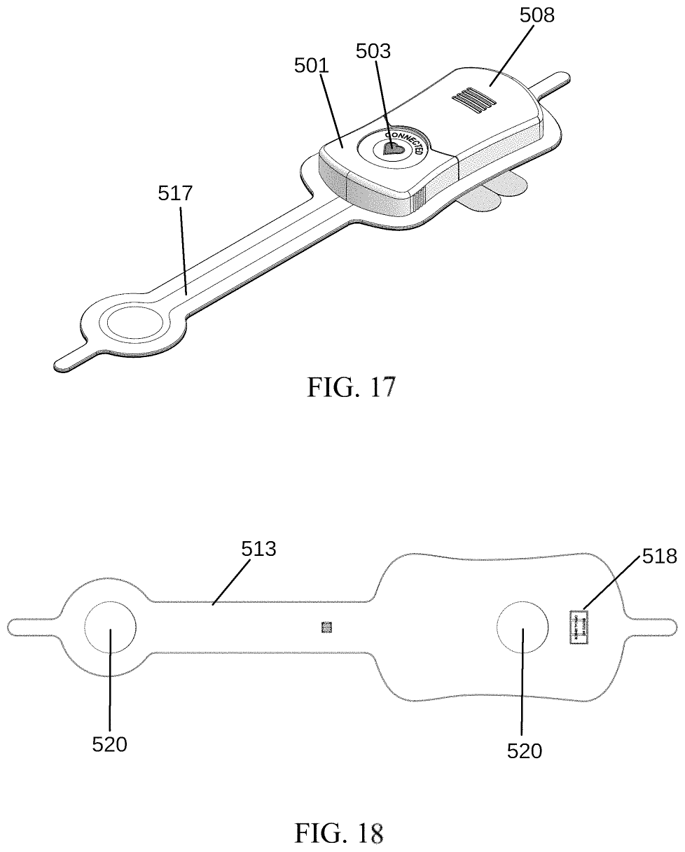

FIG. 17 depicts a top perspective of the reusable snap dome switch and electronics case connected with the disposable sensors and case in accordance with an embodiment of the present disclosure. The perspective includes the case 501 for the reusable sensor module, the snap dome switch or button 503, the master case 508 over the adhesive disposable patch, and the cardio flex circuit reference number 517.

FIG. 18 depicts a bottom perspective of the reusable snap dome switch and electronics case connected with the disposable sensors and case in accordance with an embodiment of the present disclosure. The perspective includes the master patch 513, the optical sensor 518 and the ECG electrodes 520.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A multiparameter monitor (MPM) for a patient, comprising:
a) an electrode patch (DEP) comprising a plurality of snap electrodes, an optics interface and a thermal/respiratory interface to a skin surface of the patient;
b) an electronics module and wireless transmitter (EMT) in connection with the plurality of snap electrodes and configured to transmit a signal data based on a connection and a disconnection of the plurality of snap electrodes;
c) a reflective pulse oximeter (RPO) separated from a skin surface of the patient based on a thickness of the DEP, the RPO in communication with the wireless transmitter of the EMT;
d) a mobile photoplethysmogram processor (PPG) in communication with the wireless transmitter and the optics interface and configured to detect a blood pressure and volume changes in a microvascular bed of tissue adjacent the skin surface;
e) a visual display of a correlated timeline data from the EMT, RPO and PPG in relation to a body position indicating multiple irregularities outside normal events that coincide to determine a patient's health; and
f) a triaxial accelerometer configured to determine the body position of the patient during various ambulatory events relative to resting events including a prostrate and a supine position and a standing up position in regards to patient pulse blood circulation and respiration.

2. The MPM of claim 1, wherein the PPG is further configured to filter a motion artifact in the PPG based on a distinction between a pulsating arterial blood flow and a moving venous blood flow in the patient.

3. The MPM of claim 1, wherein the wireless transmitter further comprises a Bluetooth Low Energy BLE transceiver and controller with built-in antenna preprogrammed to handle all MPM electromagnetic interactions.

4. The MPM of claim 1, wherein the EMT further comprises a user experience (UX) module including LED/switches/audio/haptic/etc.

5. The MPM of claim 1, wherein the EMT further comprises a machine learning and artificial intelligence module configured for adaptive application of the MPM to various body locations including a head, a chest, an abdomen, a thorax and a plurality of locations on limbs.

6. The MPM of claim 1, further comprising a correlated timeline data of an ECG, SPO2, body temperature, respiration and blood pressure in relation to the body position.

7. The MPM of claim 1, wherein the optics interface further defines an optical window in the DEP for the RPO to measure an oxygen content in the patient's blood perfusion.

8. The MPM of claim 1, wherein the thermal/respiratory interface further defines an area over which to measure a skin temperature and a respiration of the patient via a sensor(s).

9. The MPM of claim 1, wherein the EMT further includes an ECG driver configured to generate, receive and process signals between a male snap post electrode and a female snap receptor.

10. The MPM of claim 1, wherein the RPO further comprises an angled lens configured to vary an angle of incidence of an infrared light thereof onto the patient's skin.

11. (canceled)

12. The MPM of claim 1, wherein the EMT further comprises common statistical waveform collection while resting and while ambulatory.

13. The MPM of claim 1, wherein the EMT further comprises a respiration sensor.

14. The MPM of claim 1, further comprising a multipin connector between the DEP and the EMT and configured to provide a mechanical connection and an electrical connection there between.

15. A method for a multiparameter monitoring (MPM) of a patient, the method comprising:
a) disposing an electrode patch (DEP) comprising a plurality of snap electrodes, an optics interface and a thermal interface to a skin surface of the patient;
b) connecting an electronics module and wireless transmitter (EMT) with a plurality of snap electrodes and configured to transmit a signal data based on a connection and a disconnection of the plurality of snap electrodes;
c) separating a reflective pulse oximeter (RPO) from a skin surface of the patient based on a thickness of the DEP, the RPO in communication with the wireless transmitter of the EMT;
d) detecting blood pressure and volume changes in a microvascular bed of tissue adjacent the skin surface via a photoplethysmogram in a processor (PPG) in communication with the wireless transmitter and the optics interface,
e) visually displaying a correlated timeline data from the DEP, EMT, RPO and PPG in relation to a body position indicating multiple data irregularities outside normal events that coincide to determine a patient's health; and
determining the body position of the patient during various ambulatory events relative to various resting events via a triaxial accelerometer.

16. The method of claim 15, further comprising varying an angle of incidence of an infrared light of the RPO onto the patient's skin surface.

17. The method of claim 15, further comprising varying the separation of the RPO from the skin surface of the patient plus or minus a predetermined offset.

18. (canceled)

19. The MPM of claim 15, further comprising storing common statistical PPG waveforms while ambulatory and while resting into the EMT.

20. The MPM of claim 15, further comprising correlating a timeline data of an ECG, SPO2, body temperature, respiration and blood pressure in relation to the body position.
